(19) United States
(12) Patent Application Publication
(10) Pub. No.: US 2016/0184484 A1
(43) Pub. Date: Jun. 30, 2016

(54) LOW FRICTION COATING ON VEIN CATHETER

(71) Applicant: NANEXA AB, Uppsala (SE)

(72) Inventors: Anders JOHANSSON, Uppsala (SE); Marten ROOTH, Knivsta (SE); Jan-Otto CARLSSON, Uppsala (SE)

(21) Appl. No.: 14/896,849

(22) PCT Filed: May 28, 2014
(86) PCT No.: PCT/SE2014/050661

§ 371 Date: Dec. 8, 2015

(30) Foreign Application Priority Data
Jun. 10, 2013 (SE) 1350699-3
Oct. 25, 2013 (SE) 1351271-0

(51) Int. Cl. A61L 29/10; A61M 25/00; B05D 5/08; A61L 29/14; A61L 29/16

(52) U.S. Cl. CPC A61L 29/106; A61L 29/14; A61L 29/16; B05D 5/08; A61M 25/0009; A61M 25/0045; A61L 2420/02; A61L 2400/10; A61L 2300/10; A61L 2300/404; A61M 2025/0046

(57) ABSTRACT

The present invention relates to a method of forming a boric acid coating on a substrate surface and a vein catheter comprising said coating.

### FIELD OF THE INVENTION

The present invention relates to a method of forming a coating of boric acid on a substrate surface and a vein catheter having a cannula coated with boric acid according to said method.

### BACKGROUND

Peripheral venous catheters are plastic catheters which are placed into a peripheral vein in order to administer infusions or drugs and/or to pull blood from the patient. Common puncture sites are veins on the upper side of the wrist and vena cephalica or vena basilica which run along the forearm. The usual type of peripheral venous catheter is for single-use and the catheter is delivered sterile and pre-assembled to a unit consisting of a thin hollow steel cannula, similar to cannulas for syringes, which in turn is inserted into a thin, soft and flexible plastic tube (the actual catheter), typically of polyurethane, in such a way that only some millimeter of the steel cannula tip projects out of the opening of the catheter tube. In use, the steel cannula is run through the skin and into a vein, and when the tip is in the vein, as indicated by blood emerging through the steel cannula to a blood collection cap, the steel cannula is pulled out and discarded while the plastic tube (the catheter) remains in the vein.

For the insertion of the peripheral venous catheter to be carried out as simply as possible, it is necessary that the catheter runs relatively freely from friction within the vein and that the steel cannula also runs with low friction against the inside of the catheter. The latter has proved to be a great problem. If the cannula is not easily loosened from the catheter, there is a risk that the catheter will be drawn out of the vein when the steel cannula is pulled out of the catheter, and the patient must then be punctured again. This problem is solved today in various ways, but usually a thin film of silicone oil is applied already in the factory on the outside of the catheter as well as between the catheter and the steel cannula.

It is desired by catheter manufacturers and medical care that the silicone oil be replaced by some type of new low friction coating. As reasons why it is desired to replace the current surface treatments it is, on one hand, stated that the silicone oil enters the patient's blood stream with resulting undesired effects, such as a certain increased risk of plaque formation, on the other hand, it has been found that the silicone oil promotes formation of fungial biofilm on the inside of the catheter, thirdly, the silicon coating process results in formation of silicone vapor which will contaminate all the surrounding surfaces and finally the coating with silicone oil on the catheter cannulas means that the factory staff will be subjected hazardous vapors. Best of all would be to have a low friction coating which exhibits a similarly low friction as today's silicone oil coatings. There is therefore a need for a novel low friction material which may replace the silicone oil.

Boric acid is a known low friction material which is believed to have a low friction coefficient due to its layered crystal structure similar to the crystal structure of graphite, hexagonal boron nitride and MoS₂.

Boric acid is also used as an antiseptic for minor cuts or burns for example and may be found in lotions or creams. It may also be used as an antibacterial compound when treating acne for example and in eye drops.

U.S. Pat. No. 5,477,864 discloses the use of boric acid to provide a solid lubricant arranged on a ceramic material (Ti—Nb—Zr alloy) used for example as a guide wire in order to lower the friction between said guide wire and a catheter wall or body tissue.

However, the preparation of the boric acid surface or coating is usually performed using advanced technique demanding special coating equipment, high temperature or low pressure which makes the preparation expensive. U.S. Pat. No. 5,477,864 discloses the use of vacuum evaporation (with or without ion bombardment) or deposition of B₂O₃ on the substrate surface forming a film which will react with the water in vivo to form a boric acid layer. Some of these techniques are not only expensive but the reproducibility can be questioned.

### SUMMARY OF THE INVENTION

The object of the present invention is to provide an easy to use method for forming a boric acid coating on a surface in order to lower the friction coefficient of the surface without the use of advanced equipment.

In a first aspect the present invention relates to a method of coating a substrate surface with boric acid comprising:
providing a substrate surface;
providing a solution of a boric acid forming compound and a polar solvent;
applying the solution to the substrate surface; and
allowing the solvent to evaporate leaving an at least partly coherent coating of boric acid on the surface.

In a second aspect the present invention relates to a vein catheter device comprising a housing, a catheter tubing and a cannula, wherein
the housing has a first end and a second end arranged along a central axis and an opening at the first end of the axis;
the catheter tubing has a first end and a second end, and wherein the first end is mounted to the housing at the second end of the housing such that when the cannula is in a loading position the cannula is inserted through the opening of the housing into the catheter tubing;
the cannula has an inner surface and an outer surface, wherein the length of the cannula is such that it extends beyond the second end of the catheter tubing when the cannula is in the loading position;
   and wherein the outer surface of the cannula is at least partly coated with boric acid.

In a third aspect the present invention relates to a device coated with boric acid by the method according to the present invention.

### BRIEF DESCRIPTION OF THE FIGURES

FIG. 1, a schematic figure of a vein catheter and a safety cap.

FIG. 2, a schematic figure disclosing a safety cap, a housing with a tubular catheter and a cannula.

FIG. 3, setup cannula/catheter separation.

FIG. 4, graph from cannula/catheter separation tests.

FIG. 5, tip penetration test set-up.

FIG. 6, graph from tip penetration tests.

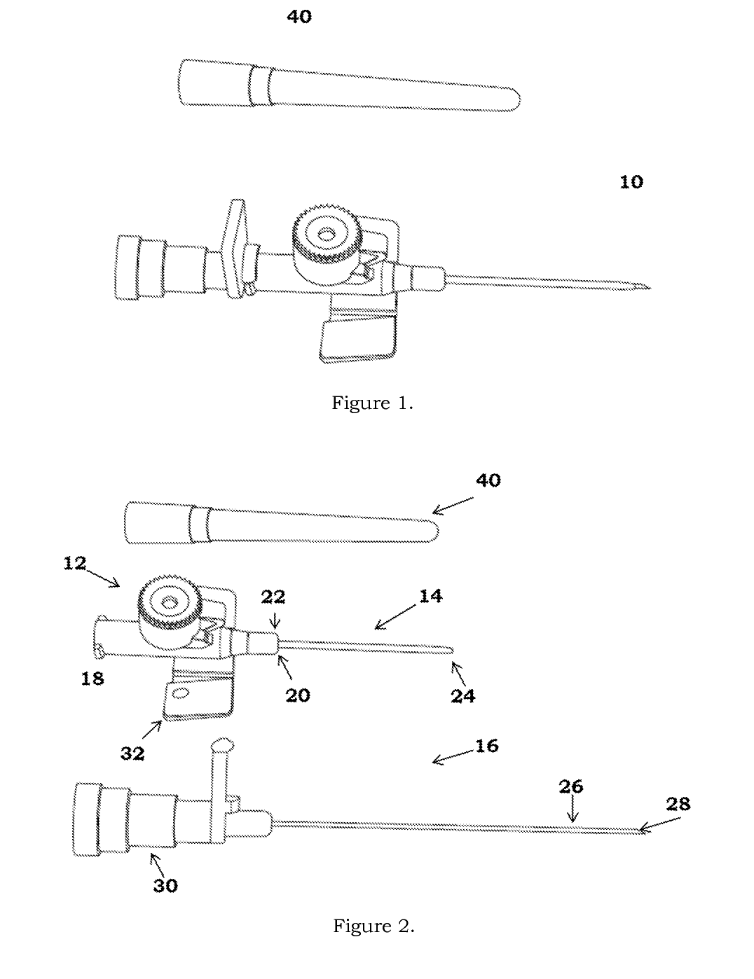

### DETAILED DESCRIPTION OF THE INVENTION

The present invention provides a solution to the problem of high friction between the cannula and the tubular catheter of a vein catheter for example which may cause the catheter to be drawn out when removing the cannula. The present invention further provides a solution to the problem associated with the use of silicon oil as described above. The use of a boric acid coating provides a low friction surface which does not come off in parts but may slowly dissolve under wet conditions and therefore there is no or minimal risk of any systemic reactions or disturbances caused by the coating. The boric acid coating has also the advantage of storage stability, i.e. the coating remains during storage in comparison with for example silicone oil coating where the silicone oil with time migrates leaving a less coated or even an uncoated surface. Also, when the tubular catheter has a tapered end the force exerted by the catheter on the cannula is higher and when silicone oil is used as coating the oil will be pressed or squeezed out leaving a less coated or even an uncoated cannula surface. Since the boric acid coating is solid it will remain even at the tapered end. Moreover, boric acid shows some well-described antiseptic properties and has previously been used in for instance eye drops. Furthermore, the boric acid coating according to the present invention also provide a coating which is very predictable concerning the force need to remove the catheter, i.e. the force needed is more or less the same in every catheter.

In order for a coating like boric acid on a consumable to be interesting the technique of preparing the coating needs to be simple and non-expensive. Furthermore, the method should provide the wanted properties of the coating and reproducible results should be obtained from the method, i.e. the obtained coating should be the same or at least very similar every time. The present invention discloses a method that is easy to use and does not demand any advanced and expansive equipment and where the obtained coating is not only reproduced every time but the structure or morphology of the coating is also very favourable. Existing production apparatus for applying silicone oil on cannulas can also be used for this application.

The method of forming a boric acid coating on a surface according to the present invention comprises providing a substrate surface, preferably a hydrophilic surface, and a solution comprising boric acid or a boric acid forming compound and a polar solvent. The solution is then applied to the substrate surface and the solvent is then allowed to evaporate leaving a coating of boric acid. The boric acid coating is preferably coherent and is at least partly covering the substrate surface, more preferably the whole surface. The crystal structure of boric acid is in the shape of layers which allows the layers to easily slide over each other during use.

The substrate surface is one embodiment a hydrophilic surface. In one embodiment the water contact angle of the substrate surface is less than 90°, or 75° or less, or 60° or less, or 50° or less. The substrate may be inorganic such as a metal, metal oxide or a ceramic. In one embodiment the substrate or the substrate surface is a metal oxide, metal sulphide or metal nitride. The metal may be for example stainless steel, titanium or alloys of Fe, Ti, Sn, Al, Cr and oxides thereof. Examples of oxides may be iron oxides or titanium oxides or chromium oxide. The substrate can also be a particle composite or a fibre composite or a polymeric substrate with a hydrophilic surface or which has been treated by conventional method to become hydrophilic. Examples of such treatments can for instance be plasma treatment, hydrolysis, aminolysis, grafting of hydrophilic compounds or treatment in a hydrogen peroxide containing solution. In one embodiment the substrate surface is cleaned prior to coating. The cleaning may be performed using any suitable solvent. In one embodiment the substrate is a needle or a cannula of a vein catheter.

Prior to applying the solution to the surface the surface may be pre-treated. The pre-treatment may for example be washing the surface in order to remove grease for example. The substrate may be oxidised for example during heat treatment or chemically or physically treated in order to increase the hydrophilicity for example by creating ionic or polar groups on the surface. These ionic or polar groups may for example be carboxyl groups, oxyl, hydroxyl groups and/or amide groups. By treating the surface with suitable acids or basis and/or heat ionic or polar groups may be provided on the substrate surface increasing the hydrophilicity of the surface.

Boric acid forming compounds may be but is not limited to boron (B), B(OH)₃, HBO₂, H₂B₄O₇, H₃BO₃ or B₂O₃ or salts of boric acid. A mixture of different boric acid forming compounds may also be used.

The solution comprises a polar solvent and a boric acid forming compound. In one embodiment the solvent comprises at least one C1-C5 alcohol. In one embodiment the solvent is non-flammable or it does not contain any flammable solvents. In another embodiment the solvent comprises a mixture of at least two C1-C5 alcohols. In yet another embodiment the solvent comprises a mixture of at least one C1-C5 alcohol and water. The C1-C5 alcohols may be methanol, ethanol, propanol, butanol or pentanol or any of their isomers for example iso-propanol. In one embodiment the solvent is selected for its low boiling point and/or high vapour pressure in order to evaporate the solvent faster and thereby obtaining a coating at a shorter time. Suitable mixtures of C1-C5 alcohols may be methanol-ethanol mixtures or methanol-propanol mixtures or ethanol-propanol mixtures. When the solvent comprises a mixture of a C1-C5 alcohol and water the weight ratio between the alcohol and water may be from 20:1 to 1:20, for example 15:1 or less, or 10:1 or less, or 5:1 or less, or 1:15 or more, or 1:10 or more, 1:5 or more. Preferred weight ratio ranges are 10:1 to 1:10 or 5:1 to 1:5. In one embodiment the solvent comprises water and a C1-C5 alcohol where the C1-C5 alcohol content is 10 to 50 weight %, preferably 20 to 40 weight %, and preferably the C1-C5 alcohol is methanol or ethanol.

In another embodiment the polar solvent is an ester, a carbonyl, an amine or amide containing solvent or a silicon based solvent. In one embodiment at least one solvent is selected from dichloromethane, tetrahydrofuran, ethyl acetate, acetone, dimethylformamide, acetonitrile, dimethyl sulfoxide, propylene carbonate, formic acid, acetic acid and nitromethane.

In one embodiment the solution is saturated or near saturated with the boric acid forming compound. A saturated solution is believed to lead to a faster coating formation and may also lead to an improved structure or morphology of the obtained coating. Furthermore, a saturated solution is easier to keep the same concentration in by using an excess of not dissolved boric acid or boric acid forming compound, i.e. if solvent evaporates the solution will maintain its concentration. In one embodiment the amount of boric acid forming compound is 70 weight % of saturation or more, or 80 weight % of saturation or more, or 90 weight % of saturation or more, or 95 weight % of saturation or more. In a further embodiment the boric acid forming compound is oversaturated in the solution.

The solution may be at room temperature when applied but could be around 0° C. or higher, or 10° C. or higher, or 50° C. or higher, or 70° C. or higher, or 120° C. or less, or 100° C. or less, or 80° C. or less. By having a solution prepared at a higher temperature and thereafter cooled the solution may be over saturated with boric acid forming compound. A solution can also be oversaturated by evaporation of the solvent.

The application may be performed in any suitable way for example by spraying the solution onto the surface or dipping the substrate into the solution. The application may be performed two or more times in order to obtain thicker coatings of the boric acid coating and/or to obtain a more coherent boric acid coating. In one embodiment the substrate surface is coated or dipped three times. By dipping the substrate into the solution and then allowing the solvent to evaporate a very easy and fast way of forming a boric acid coating is achieved. In one embodiment the application of the solution is done by dipping the substrate into the solution for a sufficient period of time, for example 1 second or more, or 10 seconds or more, or 1 minute or more. In one embodiment the substrates are removed from the solution at a speed sufficient to produce a uniform film of solution without the formation of droplets on the surface, which after evaporation of the solvent produces a uniform coating. The speed at which the substrates are removed (removal speed) also influence the solvent evaporation which in turn affects the coating properties. The removal speed may be 0.1 mm/s or more, or 0.5 mm/s or more, or 1 mm/s or more, 5 mm/s or more, or 10 mm/s or more or 15 mm/s or more, or 20 mm/s or more, or 40 mm/s or less, or 35 mm/s or less, or 30 mm/s or less, or 25 mm/s or less. Examples of ranges of removal speeds are for example 0.2 mm/s to 35 mm/s, or 0.5 to 5 mm/s, or 5 to 30 mm/s, or 15 to 25 mm/s. In one embodiment the substrates or the substrate surfaces are provided to the solution, for example by dipping, at a speed sufficient to produce an even coating. The speed at which the substrates are provided or added to the solution (adding speed) may be 5 mm/s or more, or 10 mm/s or more or 15 mm/s or more, or 20 mm/s or more, or 40 mm/s or less, or 35 mm/s or less, or 30 mm/s or less, or 25 mm/s or less. Preferred ranges of removal speeds are for example 10 to 30 mm/s, or 15 to 25 mm/s. Without being bound by theory, the adding speed and/or the removal speed is believed to influence the final crystal orientation and the coverage of the coating and thereby also the feature of low friction. It is also believed that the type of solvent or solvent mixture may influence the crystal orientation of the coating. The boric acid or the boric acid forming compound self assembles on the substrate surface in the solution, which after withdrawal and evaporation results in an arrangement where the crystal planes are substantially arranged parallel to the substrate surface. The substrate or the surface may be cleaned prior to coating.

The solvent may be evaporated by air drying, by heating and/or at reduced pressure.

The present invention further relates to a vein catheter device comprising a housing 12, a catheter tubing 14 and a cannula 16. The catheter device is also usually provided with a safety cap 40. The housing has a first end and a second end 18 and 20 arranged along a central axis and an opening at the first end of the housing and the tubing has a first and a second end 22 and 24 and where the first end is mounted to the housing at the second end of the housing. The tubing is mounted to the housing in such a way that the cannula 16 when in a loading position is inserted into the opening of the housing into the catheter tubing. The cannula, which may be metallic, has an inner surface and an outer surface 28 and 26 and the length of the cannula is such that it extends beyond the second end of the catheter tubing when the cannula is in the loading position. The outer surface of the cannula is at least partly coated with boric acid. The cannula may preferably have a handle 30. The housing may comprise more than one opening in order to facilitate multiple connections to the same catheter device, and the housing may further also comprise wings 32 stretching out perpendicularly to the axis in order to keep the housing and catheter device better in place.

In one embodiment the catheter tubing is made of any suitable polymer material. For example the tubing is made of polyethylene, polypropylene, polyvinyl chloride (PVC), polysiloxane, polyurethane or rubber or co-polymers or mixtures thereof.

The inventors of the present invention have developed a boric acid coating where the deposited boric acid exhibits a preferred orientation in the (001) crystal plane and where said plane is substantially arranged parallel to the substrate surface. This parallel arrangement increases the lubricating effect of the coating since the crystal planes of boric acid gives the lubricating effect and a parallel arrangement would further facilitate the sliding of planes over each other. At least two planes of boric acid coating are needed for this effect. Boric acid crystallizes with the (001) crystal planes on top of each other and parallel to the substrate surface. Since the chemical bonds are strong within the crystal planes but weak between the crystal planes, the (001) planes can slide on each other and become an ideal solid lubricant. Additional sliding may also occur in grain boundaries as well as in the interface between the substrate and the boric acid. The method of preparing the coating according to the present invention employs chemical self-organization to orient the crystal planes in parallel to the substrate surface thereby creating a low-friction sliding system.

In one embodiment the catheter tubing has an inner diameter and wherein the inner diameter of the second end 24 of the catheter tubing is tapered. This tapering is provided to keep the cannula in place prior to insertion into the vein. A problem with the silicone oil coating found today on vein catheters for example is that during storage the silicone oil migrates or may even be squeezed out leaving a less or even uncoated surface, which affect the low friction properties negatively. A coating according to the present invention remains even during long storage.

The present invention may be used to coat other devices such as central vein catheters, guide wires, suture threads, bone screws, K-wires etc.

The present invention has at least the following advantages:
Non-flammable solvent for dip coating
No use of silicone
Reproducible results
No post treatment of the coating is necessary
Boric acid is antiseptic
Boric acid is already in use in pharmaceutical and medical applications
Boric acid is an inexpensive compound
Reduces tip penetration
Reduces the force for cannula-catheter separation
Reduces force oscillations

### EXAMPLES

### Example 1

### Dip Coating

A saturated solution of 20.2 wt % of boric acid in methanol was prepared and a clean cannula of stainless steel was provided. The cannula was dip coated by dipping the cannula in the solution. The dipping rate, i.e. the speed at which the cannula was lowered into the solution, and the speed at which the cannula was removed from the solution was 20 mm/s. The cannula was paused at its lowest point in the dipping procedure for two seconds.

### Friction Study

Coated and uncoated cannulas were pushed through a latex cloth and the force needed to introduce the cannulas through the opening in the cloth was measured.

For uncoated cannulas a force of 0.8 N was needed while coated only needed a force of 0.2 N at a steady-state pressure.

The force needed to remove a tubular catheter from coated and uncoated cannulas was also tested. The force need from uncoated was 4.5 N and for coated 1.9 N. Furthermore, the scattering of the force needed was also much broader for uncoated cannulas, 1 N, while the scattering for coated were 0.03 N.

### Example 2

### Penetration Test and Cannula/Catheter Separation

Cannulas for peripheral venous catheters were coated with boric acid by means of a dip coating technique according to the present invention. The boric acid serves as a solid lubricant. The cannulas were thereafter tested with regards to tip penetration and cannula/catheter separation force. The tests were performed on coated and uncoated cannulas using an in-house built test equipment.

### Dip Coating

The cannulas and catheters used in these tests were parts from the peripheral venous catheters Venflon Pro 1.1. The dip coatings were performed in an in-house built automatized dip coating equipment using a non-flammable boric acid solution (methanol-water solution with 30 wt % methanol, saturated with boric acid). The cannulas were removed from the solution at a speed of 10-25 mm/min. Cannulas and catheters were considered clean as received and no additional cleaning was made. Prior to coating, the cannulas were investigated by microscopy in order to assure that the tips were undamaged.

### Cannula/Catheter Separation Force

### Test Setup and Method

30 uncoated cannulas and 30 coated cannulas were inserted into catheters and rested for 48 hours.

The dip equipment with a cannula/catheter allows control of the vertical speed at which the cannula and catheter separates. The setup is shown in FIG. 3.

The cannulas and catheters were separated (pulled apart) at a constant speed (0.1 inch/min) and a representation of the force could be seen on the scale. The display of the scale was filmed during the separated. The recorded film was analyzed frame by frame to obtain a representation in grams of the required force during the separation.

### Test Results

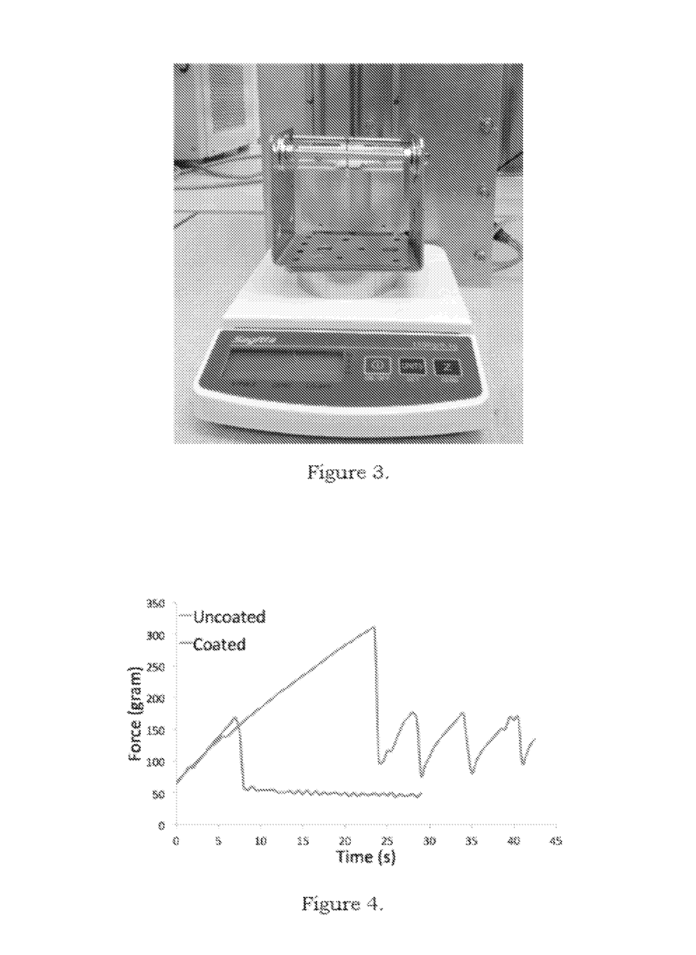

Typical graphs from cannula/catheter separation tests on coated and uncoated cannulas are shown in FIG. 4. Maximum separation force representation was noted and the results are presented in Table 1.

TABLE 1
Cannula/catheter separation force representation
on uncoated and coated samples.

Separation force

| Sample | Average | Stdev |
|---|---|---|
| Uncoated | 302.9 | 31.9 |
| Coated | 169.8 | 29.9 |

Notes:
It is clearly observed that the force oscillations after the maximum were significantly reduced on coated samples when comparing with uncoated samples.

### CONCLUSIONS

The separation force maximum was lowered by 44% by coating the samples with boric acid using the method according to the present invention. Also, the force oscillations were significantly reduced on the coated samples.

### Tip Penetration

### Test Setup and Method

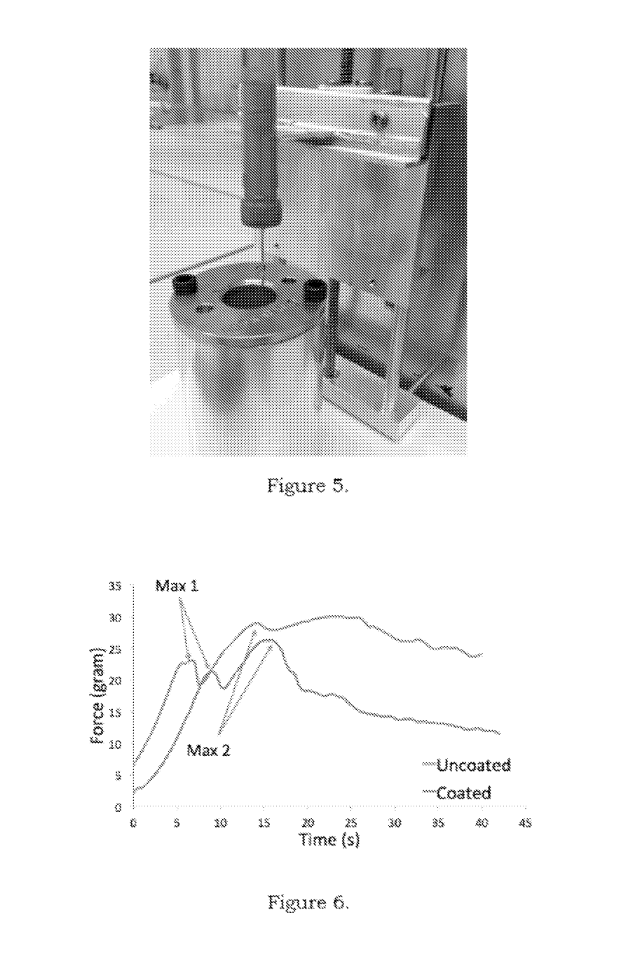

The dip coating equipment with some modifications were used for the tests. The dip equipment with a mounted cannula allowed control of the vertical speed at which the cannula moves. The setup is shown in FIG. 5.

A latex film (Dental Dam, Coltène/Whaledent Inc) was mounted in a holder and the holder was placed on a scale. The mounted latex film was penetrated on 20 spots clockwise, 3 mm from the edge.

The cannulas were vertically moved at a constant speed (0.5 inch/min) through the latex film and the display of the scale was filmed during the penetration. The recorded film was analyzed frame by frame to obtain a representation in grams of the required tip penetration force.

10 coated cannulas and 10 uncoated cannulas (reference) were tested on each latex film.

### Test Results

Typical graphs from tip penetration tests on coated and uncoated cannulas are shown in FIG. 6. Max 1 and max 2 (see FIG. 4) (Max 1 and 2 are the first and the second maximum force needed to penetrate the latex) were noted and the results are presented in Table 2. As can be seen the coating does not have a negative effect on the penetration, i.e. the sharpness of the cannula is not affected. Instead the coating reduces force oscillations.

TABLE 2
Tip penetration force representation
on uncoated and coated samples.

| | |
|---|---|
| Max 1 | Max 2 |

| Sample | Average | Stdev | Average | Stdev |
|---|---|---|---|---|
| Uncoated | 22.36 | 1.84 | 31.97 | 3.23 |
| Coated | 22.09 | 0.85 | 26.34 | 1.58 |

Notes:
We observed that there was a slight different in required tip penetration force to penetrate latex films in different mountings of the film in the holder, i.e. Max 1 values for two different mountings of latex films were for uncoated cannulas 20.83 +/− 1.17 and 22.36 +/− 1.84, respectively. That is the reason that we used 10 of each of the coated and uncoated cannulas on the same latex film.

1-16. (canceled)

17. A method of coating a substrate surface with boric acid comprising:
providing a substrate surface;
providing a solution of a boric acid forming compound and a polar solvent;
applying the solution to the substrate surface; and
allowing the solvent to evaporate leaving an at least partly coherent coating of boric acid on the surface.

18. The method of claim 17 wherein the surface is hydrophilic.

19. The method according to claim 17 wherein the substrate surface is a metal or a metal oxide.

20. The method according to claim 17 wherein the polar solvent comprises at least one C1-C5 alcohol or comprises a mixture of at least two C1-C5 alcohols or comprises a mixture of at least one C1-C5 alcohol and water.

21. The method according to claim 17 wherein the solution is saturated or near saturated with the boric acid forming compound.

22. The method according to claim 17 wherein the application of the solution is done by spraying the solution onto the substrate surface.

23. The method according to claim 17 wherein the application of the solution is done by dipping the substrate surface into the solution for a sufficient period of time and then removing the substrate surface from the solution in a rapid motion.

24. The method according to claim 17 wherein the solution is saturated with the boric acid forming compound and the polar solvent comprises a mixture of at least one C1-C5 alcohol and water and wherein the weight ratio between the alcohol and water is 5:1 to 1:5.

25. The method according to claim 23 wherein the solution is over saturated with boric acid forming compound and wherein polar solvent comprises a mixture of at least one C1-C5 alcohol and water and wherein the weight ratio between the alcohol and water is 10:1 to 1:10 and wherein the solvent is evaporated by heating and/or reduced pressure.

26. The method according to claim 17 wherein the application of the solution is repeated at least once in order to increase the thickness of the coating and/or to increase the coherency of the coating.

27. The method according to claim 23 wherein the adding speed is 10 to 30 mm/s and the removal speed is 0.2 to 30 mm/s.

28. The method according to claim 17 wherein the substrate is a cannula of a vein catheter.

29. A vein catheter device comprising a housing, a catheter tubing and a cannula, wherein
the housing has a first end and a second end arranged along a central axis and an opening at the first end of the axis;
the catheter tubing has a first end and a second end, and wherein the first end is mounted to the housing at the second end of the housing such that when the cannula is in a loading position the cannula is inserted through the opening of the housing into the catheter tubing;
the cannula has an inner surface and an outer surface, wherein the length of the cannula is such that it extends beyond the second end of the catheter tubing when the cannula is in the loading position;
and wherein the outer surface of the cannula is at least partly coated with crystal sheets of boric acid.

30. The device according to claim 29 wherein the catheter tubing is made of polyethylene, polypropylene, polyvinyl chloride or polyurethane or co-polymers thereof.

31. The device according to claim 29 wherein the sheets are substantially arranged parallel to the surface of the cannula.
